(19) United States
(12) Patent Application Publication
(10) Pub. No.: US 2006/0246153 A1
(43) Pub. Date: Nov. 2, 2006

(54) Herbal compositions for the teatment and prevention of prostate disorders

(76) Inventors: Ezio Bombardelli, Groppello Cairoli (IT); Paolo Morazzzoni, Milano (IT); Antonella Riva, Milano (IT)

Correspondence Address: YOUNG & THOMPSON, 745 SOUTH 23RD STREET, 2ND FLOOR, ARLINGTON, VA, 22202, US

(21) Appl. No.: 10/563,380

(22) PCT Filed: Jun. 17, 2004
(86) PCT No.: PCT/EP04/06550

§ 371 Date: Apr. 27, 2006

(30) Foreign Application Priority Data
Jul. 8, 2003 (IT) MI2003A001388

(51) Int. Cl. A61K 36/889; A61K 31/685; A61K 31/20; A61K 31/198; A61K 31/36; A61K 31/015; A61K 33/32; A61K 36/81

(52) U.S. Cl. 424/641; 424/727; 424/776; 514/452; 514/558; 514/561; 514/78; 514/763

(57) ABSTRACT

Disclosed are compositions comprising: a) silymarin or components thereof, in free form or complexed with phospholipids; b) lycopene, in pure form or in the form of Lycopersicum aesculentum extract; c) lauric acid or a non-toxic ester or salt thereof or the lipophilic extract of Serenoa repens; d) optionally, zinc salts and/or selenium compounds.

### FIELD OF INVENTION

This invention relates to composition of natural compounds of plant origin, and possibly oligoelements, for the treatment of prostate hypertrophy and the prevention of prostate cancer.

### BACKGROUND TO THE INVENTION

It is known that silymarin, and in particular silibinin, are compounds with anti-hepatotoxic activity (Reinhard S. et al. Drugs, 2001, 61, 2035-2063) and anti-inflammatory activity (Gupta P. O. et al. Phytomedicine, 2000, 7, 21) when administered by the topical or systemic route; this molecule is also known to have an affinity for estrogen receptors (Scambia G. et al. European J. of Cancer, 1996, 32A, 878). Silymarin has been used for decades to treat liver disease of various kinds and to treat α-amanitin and phalloidin poisoning. U.S. Pat. No. 5,714,473 also describes the use of silymarin and silibinin in modulating or reducing the toxicity of oncological drugs such as cisplatinum and anthracyclines. WO 96/37209 claims that silibinin, in the form of a complex with phospholipids, inhibits the proliferation of hormone-dependent tumours of the ovary and breast, and has synergic effects with platinum complexes. Its affinity for estrogen receptors enables the molecule to accumulate in sites which abnormally express estrogen receptors, performing its particular antioxidant, anti-inflammatory and antiproliferative effects on the organ that over-expresses them. These anti-inflammatory and antiproliferative effects are particularly important in the treatment and prevention of non-hormone-dependent prostate tumours, for the reasons described below.

In vitro, silymarin and especially silibinin inhibit the proliferation of independent androgenic prostate cancer cells, thus arresting the cell cycle in G1.

Lycopene is a lipophilic antioxidant which, as widely known, has a preventive effect on the genesis of prostate cancer. At epidemiological level there is an inverse correlation between lycopene plasma levels and prostate tumours, for reasons which are not yet fully understood; this procarotenoid, which does not generate vitamin A in the body, enters the lipoprotein, where it inhibits cholesterol oxidation, and said inhibition may influence the synthesis and metabolism of steroid hormones. In experiments conducted on patients with localised prostate adenocarcinoma awaiting surgical eradication, lycopene, taken as part of the diet for three weeks at the dose of 28 mg a day, reduced the plasma levels of PSA (prostate-specific antigen), and greatly reduced oxidative damage to the DNA of the post-operative biopsy tissue (J Natl Cancer Inst 2001, 93, 1872-79).

Finally, the lipophilic extract of Serenoa repens has been used for some time in the treatment of benign prostate hypertrophy.

### DESCRIPTION OF THE INVENTION

It has now surprisingly been found that composition of:
a. silymarin or components thereof, in free form or complexed with phospholipids;
b. lycopene, used in pure form or in the form of Lycopersicum aesculentum extract;
c. lauric acid or a non-toxic ester or salt thereof or the lipophilic extract of Serenoa repens;
d. and optionally, zinc salts and/or selenium compounds, reduce cell proliferation; prostate hyperplasia, PSA and oxidative damage to the DNA, to a far greater extent than was known for the ingredients taken separately.

Silymarin or its main components (silibinin, silidianin and silichristin, especially silibinin), which are extracted from milk thistle (Silybum marianum), can be used as such or in the form of complexes with phospholipids, as disclosed in EP 0209038.

The complex of silibinin with phosphatidylcholine is particularly preferred.

Lycopersicum aesculentum extract can be prepared as described in EP 0818225, PCT/EP03/02749, while Serenoa repens extract can be prepared as disclosed in EP 0250953.

The lauric acid is preferably in the form of a methyl or ethyl ester or zinc salt.

Adducts of selenium with different non-toxic substrates can be used as a source of selenium in order to administer 5 to 20 micrograms of selenium. Methylselenocysteine is particularly preferred.

The various ingredients are preferably formulated as tablets, hard or soft gelatin capsules or drinkable formulations, with suitable excipients.

The average daily doses of the various ingredients range between 100 mg and 1 g for silibinin, preferably 150-300 mg; 2 to 30 mg for lycopene, preferably 7.5 mg; and 20 to 80 mg for lauric acid or its non-toxic esters or salts, preferably 40 mg; zinc is administered in amounts of between 8 and 16 mg, preferably 12 mg; and selenium, in the form of methylselenocysteine, in amounts of between 5 and 20 micrograms a day, preferably 10 micrograms.

In the case of the phospholipid complexes of silibinin or silymarin, the doses refer to the active ingredients content.

A preferred composition contains 160 mg of silibinin complexed with phosphatidylcholine, 7.5 mg of lycopene, 22 mg of Zn laurate and 12 μg of methylselenocysteine.

The various ingredients are diluted with suitable excipients which ensure acceptable absorption of the total formulation. Using these compositions, the symptoms of benign prostate hyperplasia such as dysuria and daytime and nocturnal pollakiuria, and the progress of prostate enlargement, have been reduced in prostate patients. In patients with non-hormone-dependent prostate cancer, this combination reduced the plasma PSA values, indicating a direct effect on cell proliferation.

The following examples illustrate the invention in detail.

### EXAMPLE 1

Capsules containing:

| | |
|---|---|
| Silymarin complex with phosphatidylcholine | 240 mg |
| Serenoa repens extract | 200 mg |
| Tomato extract with 10% lycopene | 50 mg |

### EXAMPLE 2

Capsules containing:

| | |
|---|---|
| Silybin complex with phosphatidylcholine | 160 mg |
| Lycopene | 20 mg |
| Zn laurate | 30 mg |
| Methylselenocysteine | 0.01 mg |

1. Compositions comprising:
a. silymarin or components thereof, in free form or complexed with phospholipids;
b. lycopene, in pure form or in the form of Lycopersicum aesculentum extract;
c. lauric acid or a non-toxic ester or salt thereof or the lipophilic extract of Serenoa repens;
d. optionally, zinc salts and/or selenium compounds.

2. Compositions as claimed in claim 1, in which component a. is silybin complexed with phosphatidylcholine.

3. Compositions as claimed in claim 1, in which component c. is lauric acid methyl or ethyl ester or its zinc salt.

4. Compositions as claimed in claim 1 in which the selenium compound is methylselenocysteine.

5. Compositions as claimed in claim 1 containing daily dosages of 100 mg to 1 g of silybin o silymarin o the equivalent of the corresponding phospholipid complexes; 2 to 30 mg of Lycopene; 20 to 80 mg of lauric acid or esters or salts thereof; 8 to 16 mg of zinc and 5 to 20 μg of selenium as methylselenocysteine.

6. Compositions as claimed in claim 5 containing 160 mg of silybin complexed with phosphatidylcholine; 7.5 mg of Lycopene; 22 mg of Zn laurate and 12 μg of methylselenocysteine.

7. Method of preparing medicaments or nutraceuticals for the treatment of benigne prostate hyperplasy and for the treatment and the prevention of the non-hormone-dependent prostate carcinoma, which comprises: admixing
a. silymarin or components thereof, in free form or complexed with phospholipids;
b. lycopene, in pure form or in the form of Lycopersicum aesculentum extract;
c. lauric acid or a non-toxic ester or salt thereof or the lipophilic extract of Serenoa repens; and
d. optionally zinc salts and/or selenium compounds.

8. Method for treating benigne prostate hyperplasy and of treating and preventing the non-hormone prostate carcinoma, which comprises administering to a subject in need thereof an effective amount of a composition according to claim 1.
